(19) United States
(12) Patent Application Publication
(10) Pub. No.: US 2006/0094006 A1
(43) Pub. Date: May 4, 2006

(54) Immunotherapy regimens in hiv-infected patients

(76) Inventors: Genoveffa Franchini, Washington, DC (US); James Tartaglia, Ontario (CA)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/513,257

(22) PCT Filed: May 1, 2003
(86) PCT No.: PCT/US03/13737

§ 371 Date: Oct. 7, 2005

Related U.S. Application Data
(60) Provisional application No. 60/377,493, filed on May 1, 2002.

(51) Int. Cl. C12Q 1/70; C12Q 1/68

(52) U.S. Cl. 435/6; 435/5

(57) ABSTRACT

This invention relates to an improved method of maintaining an immuno-protective response in persons infected with a retrovirus during early infection or after highly active anti-retroviral therapy.

### FIELD OF THE INVENTION

This invention relates to an improved method of maintaining an immuno-protective response in persons who are infected with a retrovirus. More specifically, the invention relates to method to potentiate an immune response in persons who are at an early stage of retroviral infection, and/or who have undergone highly active anti-retroviral therapy (HAART).

### BACKGROUND

Recombinant pox virus vaccines, e.g., NYVAC- and ALVAC-based vaccines for HIV-1 have been tested in preclinical trials using either HIV-2 or SIV Gag, Pol, and Env genes in macaque monkeys (see, e.g., Benson et al., J. Virol. 72:4170-4182, 1998; Abimiku et al., J. Acquir. Immune Defic. Synd. Hum. Retrovirol. 15:S78-S85, 1997; Myagkikh et al., AIDS Res. Hum. Retroviruses 12:985-991, 1996; and Hel et al., Nat. Med. 16:1140-1146, 2000). Results from these early studies indicated that, while these vaccines do not protect from infection, they significantly reduce the viral replication within a few weeks from exposure in approximately 50% of the animals. In the case of NYVAC-SIV vaccination, the regimen changed the natural course of SIV251 infection.

In the macaque animal model, the addition of monomeric gp120 protein administered as a boost in conjunction with ALVAC-SIVgpe did not appear to improve the level of protection. (see, e.g., Pal et al., Abstract for “HIV/AIDS Vaccine Development Workshop,” Paris, France, May 5-6, 2000). These studies also suggested that more than three immunizations with NYVAC-SIV/ALVAC-SIV may not further increase the pool of memory cells, and that the vector immunity against vaccinia protein may blunt the response to SIV antigens.

Various other prime boost immunization strategies against HIV have been proposed (see, e.g., Barnett et al., AIDS Res. and Human Retroviruses Volume 14, Supplement 3, 1998, pp. S-299-S-309 and Girard et al., C R Acad. Sci III 322:959-966, 1999 for reviews). DNA immunization, when used in a boosting protocol with modified vaccinia virus Ankara (MVA) or with a recombinant fowl pox virus (rFPV) in the macaque model, has been shown to induce CTL responses and antibody responses (see, e.g., Hanke et al., J. Virol. 73:7524-7532, 1999; Hanke et al., Immunol. Letters 66:177-181; Robinson et al., Nat. Med. 5:526-534, 1999), but no protection from a viral challenge was achieved in the immunized animals. DNA immunization followed by administration of another highly attenuated poxvirus has also been tested for the ability to elicit IgG responses, but the interpretation of the results is hampered by the fact that serial challenges were performed (see, e.g., Fuller et al., Vaccine 15:924-926, 1997; Barnett et al., supra). In contrast, in a murine model of malaria, DNA vaccination used in conjunction with a recombinant vaccinia virus was promising in protecting from malaria infection (see, e.g., Sedegah et al., Proc. Natl. Acad. Sci. USA 95:7648-7653, 1998; Schneider et al., Nat. Med. 4:397-402, 1998).

Other prime boost strategies for the treatment of HIV infection have been described in WO01/82964. In these methods, immunogenicity of a recombinant poxvirus-based vaccine is enhanced by administering a nucleic acid, e.g., a DNA vaccine, to stimulate an immune response to the HIV antigens provided in the poxvirus vaccine, and thereby increase the ability of the recombinant pox virus, e.g., NYVAC or ALVAC, to expand a population of immune cells. Individuals who are treated with such a vaccine regimen may be at risk for infection with the virus or may have already been infected.

Treatment of HIV-infected individuals who have received HAART therapy has also been described (see, e.g., WO01/08702). After HIV infection, HAART treatment can sufficiently restore a patient's immune system to effectively mount a CD8⁺ response when a patient is provided with a CD8⁺CD4⁺-inducing vaccine, e.g., a DNA vaccine or a pox virus vaccine. This response can effectively maintain a low titer of virus and significantly reduce the patient dependency on HAART when the CD8⁺-inducing vaccine is an HIV vaccine.

As described above, treatment of HIV infected individuals continues to be problematic due to continued infection following highly active anti-retroviral therapy. Accordingly, what is needed are treatment schemes that promote the maintenance of an immuno-protective response in persons who are infected with a retrovirus after highly active anti-retroviral therapy (HAART).

### SUMMARY OF THE INVENTION

These and other needs are provided by the invention described herein. The current invention provides methods to improve the efficacy of protocols for the treatment of retroviral infection. The methods of the invention may be used to increase the presence of memory cells that can act to decrease the viral load in a retrovirus infected patient who has undergone highly active anti-retroviral therapy (HAART). Accordingly, the invention may be used to increase the time between HAART sessions that are necessary to maintain a retrovirus infected patient. This offers the advantage that adverse reactions to HAART may be reduced or avoided. The invention may also be used to reduce the viral load in a patient who is able to mount an immune response, but who has not undergone HAART. Such a situation may occur at an early point following infection. Accordingly, the invention may be used to extend the time between initial infection and treatment with HAART.

In one example, the invention provides a step of administering an agent that blocks HIV infectivity, e.g., an HIV entry inhibitor, a neutralizing antibody, a peptide or small molecule that blocks HIV binding to its receptor, or a cytotoxic immunoconjugate, to control viremia. The neutralizing antibody or other receptor binding blocking agent is administered to a patient who has undergone HAART therapy, has a CD4⁺ T-cell count of about 300 cells/ml or greater, and has been vaccinated with a nucleic acid or viral vaccine that stimulates CD4⁺, CD8⁺, or both CD4⁺ and CD8⁺ responses. Preferably, the individual has been vaccinated with a nucleic acid vaccine. More preferably, the individual has been vaccinated with a viral vaccine. Most preferably, the individual has been vaccinated with both a nucleic acid vaccine and a viral vaccine.

In another example, the invention provides the step of administering an immunotoxin to a patient who has undergone HAART therapy. Preferably the immunotoxin is administered to the patient following administration of a nucleic acid vaccine. More preferably, the immunotoxin is administered to the patient following administration of a viral vaccine. Most preferably, the immunotoxin is administered to the patient following administration of a nucleic acid vaccine and a viral vaccine.

The present invention is also directed to a method of stimulating an immune response and controlling viremia in a human infected with a retrovirus, e.g., HIV, who has a viral load of less than 10,000 viral copies, often 5,000 or 2,000 or less, per ml of plasma and a CD4⁺ cell count that is above 300 cells/ml, typically above about 400 cells/ml; and who has been treated with one or more anti-viral agents, which contributed to a lower viral copy and higher CD4⁺ cell count than before treatment. In one example, the method comprises administering at least one nucleic acid vaccine or viral vaccine which enters the cells and intracellularly produces HIV-specific peptides that can be presented on the cell's MHC class I molecules in an amount sufficient to stimulate a protective CD8⁺ response, and administering an agent that blocks HIV infectivity, e.g., an HIV entry inhibitor, a cytotoxic immunoconjugate that targets HIV-infected cells, a neutralizing antibody, or a compound, such as a peptide that inhibits fusion of HIV-infected cells. In another example, the method comprises administering at least one nucleic acid vaccine or viral vaccine which produces HIV-specific peptides that can be presented on the cell's MHC class II molecules in an amount sufficient to stimulate a protective CD4⁺ response, and administering an agent that blocks HIV infectivity, e.g., an HIV entry inhibitor, a cytotoxic immunoconjugate that targets HIV-infected cells, a neutralizing antibody, or a compound, such as a peptide that inhibits fusion of HIV-infected cells.

Preferably, the patient is administered both a nucleic acid vaccine, i.e. a vaccine in which the nucleic acid is not encapsidated in a virus, and an attenuated recombinant virus. A preferred virus is an attenuated pox virus, particularly NYVAC and ALVAC, attenuated vaccinia and canarypox viruses respectively. Other attenuated pox viruses such as MVA can also be used.

The vaccine can further comprise an adjuvant and may be administered a second time. The vaccine can also comprise a cytokine, such as interleukin-2 (IL-2), interleukin-7 (IL-7), interleukin-12 (IL-12), interleukin-15 (IL-15), macrophage colony stimulating factor (GM-CSF), interferon beta (IFNβ), and/or CD40 ligand in an amount that is sufficient to potentiate a CD4⁺ or CD8⁺ response.

The method of the invention can be particularly useful for a person who has been infected with HIV and has demonstrated repeated and sustained proliferative T-cell responses to gp120 envelope protein or both gp 120 envelope and p24 gag antigen.

The method of the invention can also be useful for a person who has CD4⁺ cells that are able to proliferate. Preferably, the person has a CD4⁺ T-cell count of about 300 cells/ml or greater. More preferably, the person has a CD4⁺ T-cell count of about 400 cells/ml or greater. Most preferably, the person has a CD4⁺ T-cell count of about 500 cells/ml or greater.

### BRIEF DESCRIPTION OF THE FIGURES

FIG. 1: Immune correlates of viremia containment. Plasma virus level and kinetics of neutralizing antibody appearance in blood of vaccinated macaques. SIVmac251-infected macaques were treated with continuous antiretroviral therapy (ART), which consisted of a drug regimen of nucleotide analogs. Animals were vaccinated with or without simultaneous administration of low dose IL-2 with a NYVAC vaccine that expressed the SIVmac structural gag-pol-env genes, and a chimeric fusion protein derived from the rev-tat-nef regulatory genes. Vaccination expanded both virus-specific CD4⁺ and CD8⁺ T cell responses. Production of neutralizing antibodies following cessation of ART is shown. The inset in the figure represents the frequency of cells/10⁶ producing IFN-γ following stimulation with specific peptides. Detection of anti-SIVmac251 binding and neutralizing antibodies: neutralizing antibodies (NAbs) against the primary SIVmac251/561L (produced in human PBMC) were detected as described (J. Benson, et al., J. Virol. 72, 4170 (1998).). NAb titers were defined as the reciprocal plasma dilution at which 50% of the target cells were protected from virus induced killing as detected by neutral red uptake. CEMx174 cells were used as targets for SIVmac251 virus, CEMxR5 were used as targets for SIVmac251/561L virus.

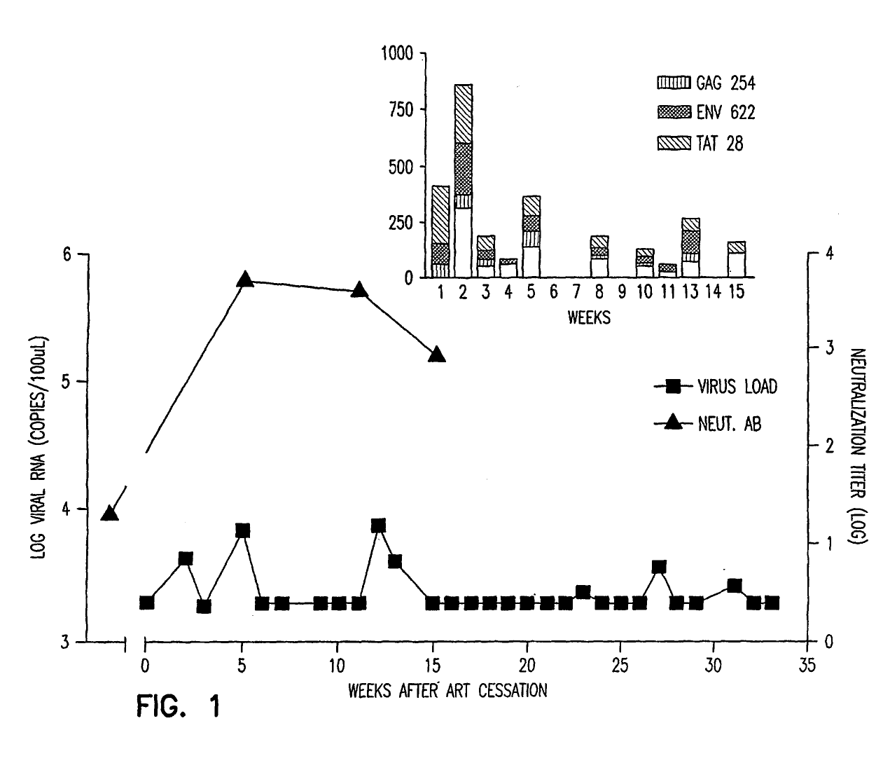

FIG. 2: Functional virus-specific T-cell responses induced by vaccination. A: Frequency of Gag181-189 CM9 tetramer-positive cells in blood of the immunized macaques. Top: DNA/FP-immunized macaques. Bottom: FP-immunized macaques. FP=Fowlpox SIV vaccine. “DNA” refers to a DNA SIV vaccine.

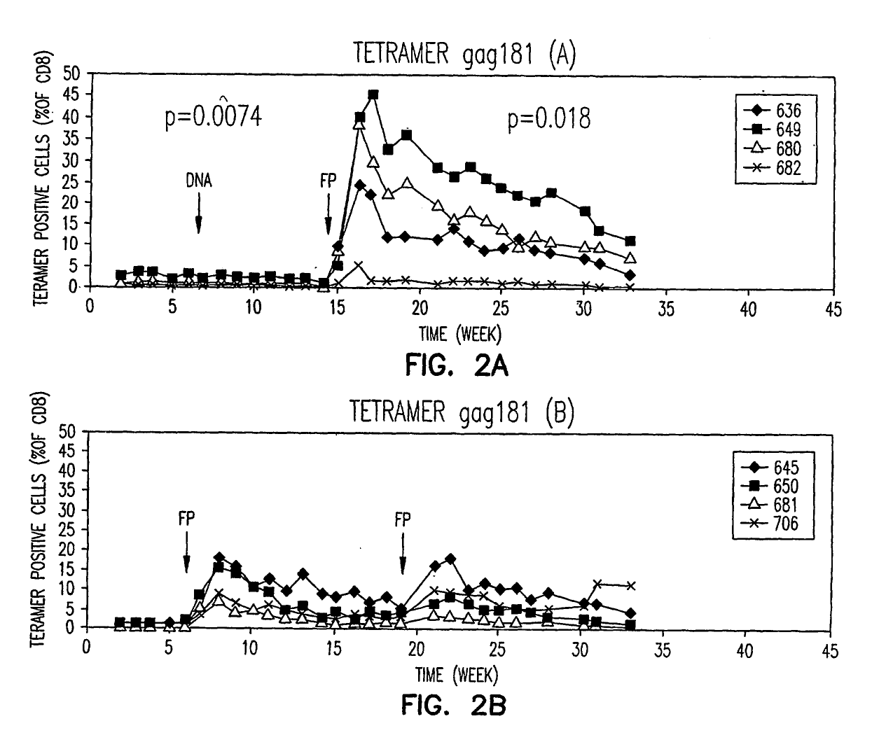

FIG. 3: Schematic representation of a treatment regimen in HIV-1-infected individuals during HAART and during HAART cessation.

### Definitions

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

“Anti-viral agent” refers to a medicament that is administered to a patient to treat viral infection. Anti-viral agents may act through numerous mechanism. For example, an anti-viral agent may reduce or eliminate viral replication, entry of a virus into a cell, or inhibit the ability of a host cell to support viral replication. Numerous anti-viral agents are known in the art and have been described (Merck Index, 13th edition, Whitehouse Station, N.J.).

“Attenuated recombinant virus” refers to a virus that has been genetically altered by modern molecular biological methods, e.g. restriction endonuclease and ligase treatment, and rendered less virulent than wild type, typically by deletion of specific genes or by serial passage in a non-natural host cell line or at cold temperatures.

“Efficient CD8⁺ response” is referred to as the ability of cytotoxic CD8⁺ T-cells to recognize and kill cells expressing foreign peptides in the context of a major histocompatibility complex (MHC) class I molecule.

“Efficient CD8⁺ T-cell response” is referred to as the ability of CD4⁺ T-cells to provide help to CD8⁺ T-cells and B-cells in the context of major histocompatibility complex (MHC) class II presentation.

An “epitope” is the portion of an antigen that is recognized and bound by an antibody, or the combination of a T-cell receptor and a major histocompatibility complex.

“Nonstructural viral proteins” are those proteins that are needed for viral production but are not necessarily found as components of the viral particle. They include DNA binding proteins and enzymes that are encoded by viral genes but which are not present in the virions. Proteins are meant to include both the intact proteins and fragments of the proteins or peptides which are recognized by the immune cell as epitopes of the native protein.

“Nucleic acid-based vaccines” include both naked DNA and vectored DNA (within a viral capsid) where the nucleic acid encodes B-cell and T-cell epitopes and provides an immunoprotective response in the person being vaccinated.

A “nucleic acid vaccine” or “DNA vaccine” as used herein refers to a nucleic acid that is not contained within a viral capsid.

“Plasma” refers to the fraction of whole blood resulting from low speed centrifugation of EDTA- or heparin-treated blood.

“Potentiating” or “enhancing” an immune response means increasing the magnitude and/or the breadth of the immune response, i.e., the number of cells induced by a particular epitope may be increased and/or the numbers of epitopes that are recognized may be increased (“breadth”). A 5-fold, often 10-fold or greater, enhancement in both CD8⁺ and CD4⁺ T-cell responses is obtained with administration of a combination of nucleic acid/recombinant virus vaccines compared to administration of either vaccine alone.

“Pox viruses” are large, enveloped viruses with double-stranded DNA that is covalently closed at the ends. Pox viruses replicate entirely in the cytoplasm, establishing discrete centers of viral synthesis. Their use as vaccines has been known since the early 1980's (see, e.g. Panicali, D. et al. “Construction of live vaccines by using genetically engineered pox viruses: biological activity of recombinant vaccinia virus expressing influenza virus hemagglutinin”, Proc. Natl. Acad. Sci. USA 80:5364-5368, 1983).

A “retrovirus” is a virus containing an RNA genome and an enzyme, reverse transcriptase, which is an RNA-dependent DNA polymerase that uses an RNA molecule as a template for the synthesis of a complementary DNA strand. The DNA form of a retrovirus commonly integrates into the host-cell chromosomes and remains part of the host cell genome for the rest of the cell's life.

“Structural viral proteins” are those proteins that are physically present in the virus. They include the capsid proteins and enzymes that are loaded into the capsid with the genetic material. Because these proteins are exposed to the immune system in high concentrations, they are considered to be the proteins most likely to provide an antigenic and immunogenic response. Proteins are meant to include both the intact proteins and fragments of the proteins or peptides which are recognized by the immune cell as epitopes of the native protein.

“TCID₅₀” refers to the median tissue culture infective dose. This is a quantity of virus that will produce a cytopathic effect in fifty percent of the cultures inoculated.

“Viral load” is the amount of virus present in the blood of a patient. Viral load is also referred to as viral titer or viremia. Viral load can be measured in variety of standard ways.

### DETAILED DESCRIPTION

This invention is a novel therapeutic modality for treating persons infected with a lymphotropic or immune destroying retrovirus. A physician presented with a patient whose immune system is compromised by retroviral infection can elect to treat that patient with a host of powerful antiviral agents including inhibitors of viral proteases, integrase, and reverse transcriptase. This is known as highly active anti-retroviral therapy (HAART). The conventional HAART protocols are complex and difficult for patients to follow. The drugs also have a number of problematic side effects. In addition, these expensive and complicated treatments do not eliminate the virus, but merely hold the virus in check. If the patient is non-compliant, the viral counts rebound. Accordingly, for the vast majority of patients, a lifetime of drugs is advised.

This invention is the discovery of a vaccine regimen for an HIV-infected patient who has a sufficient immune system to effectively mount a CD4⁺ or a CD8⁺ response when the patient is provided with a nucleic acid-based vaccine, e.g., a viral vaccine such as a recombinant pox virus vaccine or a DNA vaccine, that induces the CD4⁺ or CD8⁺ response. The patient's immune system may have been restored through use of a therapeutic protocol such as HAART. Alternatively, the patient may be at an early stage in viral infection such that they are still able to mount an effective immune response. A viral vaccine is typically administered in conjunction with a non-viral nucleic acid vaccine, e.g., a DNA vaccine, that potentiates the immune response. Following vaccination, the patient may be additionally treated with an agent that blocks HIV infectivity, e.g., an HIV entry inhibitor, neutralizing antibodies, or agents that block viral binding to its cell surface receptor. Preferably, the vaccination regimen comprises administration of multiple doses of a DNA vaccine followed by administration of multiple doses of a poxvirus vaccine.

The invention may be used to increase the number of memory cells that act in reducing viral load, thus extending the time that a patient can wait before undergoing repeated HAART sessions. Furthermore, it is thought that by blocking reinfection by the HIV virus following HAART, the invention will allow a patient to achieve a sustainable viral load of between zero and ten thousand copies of viral RNA per milliliter of serum, and more preferably between zero and five thousand copies of viral RNA per milliliter of serum in the absence of ART. This is thought to offer a tremendous advantage over present methods for treating HIV infection in which reinfection by HIV following HAART suspension may produce an equivalent or higher viral load that at the start of HAART.

### Vaccines of Use in this Invention

Vaccines useful for the induction of CD4⁺ and CD8⁺ T-cell responses comprise nucleic acid-based vaccines (delivered via a viral vector or as nucleic acid vectors that are not contained within a viral particle) that provide for the intracellular production of viral-specific peptide epitopes that are presented on MHC Class I and Class II molecules and subsequently induce an immunoprotective cytotoxic T lymphocyte (CTL) response or a helper T-cell response.

The invention contemplates single or multiple administrations of a nucleic acid-based vaccine, such as a naked DNA vaccine, or as a recombinant virus vaccine, such as a poxvirus vaccine, or preferably, both. This vaccination regimen may be complemented with administration of recombinant protein vaccines (infra), or may be used with additional vaccine vehicles.

Attenuated Recombinant Viral Vaccines

Attenuated recombinant viruses that express retrovirus specific epitopes are of use in this invention. Attenuated viruses are modified from their wildtype virulent form to be either symptomless or weakened when infecting humans. Among the recombinant viruses of use are adenoviruses, adeno-associated viruses, retroviruses, poxviruses, and other live vector-based vaccine candidates.

A recombinant, attenuated virus for use in this invention as a vaccine is a virus wherein the genome of the virus is defective with respect to a gene essential for the efficient production of, or essential for the production of, infectious virus. The mutant virus acts as a vector for an immunogenic retroviral protein by virtue of the virus encoding foreign DNA. This provokes or stimulates a cell-mediated CD8⁺ response or a CD4⁺ response.

The virus is then introduced into a human vaccine by standard methods for vaccination of live vaccines. A live vaccine of the invention can be administered at, for example, about 10⁴-10⁸ organisms/dose, or 10⁶ to 10⁹ pfu per dose. Actual dosages of such a vaccine can be readily determined by one of ordinary skill in the field of vaccine technology.

Examples of viral expression vectors include adenoviruses as described in M. Eliot et al., “Construction of a Defective Adenovirus Vector Expressing the Pseudorabies Virus Glycoprotein gp50 and its Use as a Live Vaccine”, J. Gen. Virol., 71(10):2425-2431 (October, 1990).), adeno-associated viruses (see, e.g., Samulski et al., J. Virol. 61:3096-3101 (1987); Samulski et al., J. Virol. 63:3822-3828 (1989)), papillomavirus, Epstein Barr virus (EBV) and Rhinoviruses (see, e.g., U.S. Pat. No. 5,714,374). Human parainfluenza viruses are also reported to be useful, especially JS CP45 HPIV-3 strain. The viral vector may be derived from herpes simplex virus (HSV) in which, for example, the gene encoding glycoprotein H (gH) has been inactivated or deleted. Other suitable viral vectors include retroviruses (see, e.g., Miller, Human Gene Ther. 1:5-14 (1990); Ausubel et al., Current Protocols in Molecular Biology).

The poxviruses are of preferred use in this invention. There are a variety of attenuated poxviruses that are available for use as a vaccine against HIV. These include attenuated vaccinia virus, cowpox virus and canarypox virus. In brief, the basic technique of inserting foreign genes into live infectious poxvirus involves a recombination between pox DNA sequences flanking a foreign genetic element in a donor plasmid and homologous sequences present in the rescuing poxvirus as described in Piccini et al., Methods in Enzymology 153, 545-563 (1987). More specifically, the recombinant poxviruses are constructed in two steps known in the art and analogous to the methods for creating synthetic recombinants of poxviruses such as the vaccinia virus and avipox virus described in U.S. Pat. Nos. 4,769,330, 4,722,848, 4,603,112, 5,110,587, and 5,174,993, the disclosures of which are incorporated herein by reference.

First, the DNA gene sequence encoding an antigenic sequence, such as a known T-cell epitope, is selected to be inserted into the virus. The sequence is placed into an E. coli plasmid construct into which DNA homologous to a section of DNA of the poxvirus has been inserted. Separately, the DNA gene sequence to be inserted is ligated to a promoter. The promoter-gene linkage is positioned in the plasmid construct so that the promoter-gene linkage is flanked on both ends by DNA homologous to a DNA sequence flanking a region of pox DNA containing a nonessential locus. The resulting plasmid construct is then amplified by growth within E. coli bacteria.

Second, the isolated plasmid containing the DNA gene sequence to be inserted is transfected into a cell culture, e.g. chick embryo fibroblasts, along with the poxvirus. Recombination between homologous pox DNA in the plasmid and the viral genome respectively, gives a poxvirus modified by the presence, in a nonessential region of its genome, of foreign DNA sequences.

Attenuated recombinant pox viruses are a preferred vaccine. A detailed review of this technology is found in U.S. Pat. No. 5,863,542 which is incorporated by reference herein. Representative examples of recombinant pox viruses include ALVAC, TROVAC, NYVAC, and vCP205 (ALVAC-MN120TMG). These viruses were deposited under the terms of the Budapest Treaty with the American Type Culture Collection (ATCC), 12301 Parklawn Drive, Rockville, Md., 20852, USA: NYVAC under ATCC accession number VR-2559 on Mar. 6, 1997; vCP205 (ALVAC-MN120TMG) under ATCC accession number VR-2557 on Mar. 6, 1997; TROVAC under ATCC accession number VR-2553 on Feb. 6, 1997 and, ALVAC under ATCC accession number VR-2547 on Nov. 14, 1996.

NYVAC is a genetically engineered vaccinia virus strain generated by the specific deletion of eighteen open reading frames encoding gene products associated with virulence and host range. NYVAC is highly attenuated by a number of criteria including: i) decreased virulence after intracerebral inoculation in newborn mice, ii) inocuity in genetically (nu⁺/nu⁺) or chemically (cyclophosphamide) immunocompromised mice, iii) failure to cause disseminated infection in immunocompromised mice, iv) lack of significant induration and ulceration on rabbit skin, v) rapid clearance from the site of inoculation, and vi) greatly reduced replication competency on a number of tissue culture cell lines including those of human origin.

TROVAC refers to an attenuated fowlpox that was a plaque-cloned isolate derived from the FP-1 vaccine strain of fowlpox virus which is licensed for vaccination of 1 day old chicks.

ALVAC is an attenuated canarypox virus-based vector that was a plaque-cloned derivative of the licensed canarypox vaccine, Kanapox (Tartaglia et al., AIDS Res Hum Retroviruses 8:1445-7 (1992)). ALVAC has some general properties which are the same as some general properties of Kanapox. ALVAC-based recombinant viruses expressing extrinsic immunogens have also been demonstrated efficacious as vaccine vectors. This avipox vector is restricted to avian species for productive replication. In human cell cultures, canarypox virus replication is aborted early in the viral replication cycle prior to viral DNA synthesis. Nevertheless, when engineered to express extrinsic immunogens, authentic expression and processing is observed in vitro in mammalian cells and inoculation into numerous mammalian species induces antibody and cellular immune responses to the extrinsic immunogen and provides protection against challenge with the cognate pathogen. An example of an ALVAC based vaccine is ALVAC-SIV which is engineered to express the gag, pol, and env genes of SIV_{mac251(K6W)} (Franchini et al., Nature, 328:539 (1987)) from the I3L and the H6 promoters (Perkus et al., J. Tissue Cult. Methods, 15:72 (1993)). The H6 env and the I3L gag and pol cassettes were inserted in the ALVAC C3 locus in a head-to-head (5′-to-5′) configuration.

NYVAC, ALVAC and TROVAC have also been recognized as unique among all poxviruses in that the National Institutes of Health (“NIH”) (U.S. Public Health Service), Recombinant DNA Advisory Committee, which issues guidelines for the physical containment of genetic material such as viruses and vectors, i.e., guidelines for safety procedures for the use of such viruses and vectors which are based upon the pathogenicity of the particular virus or vector, granted a reduction in physical containment level: from BSL2 to BSL1. No other poxvirus has a BSL1 physical containment level. Even the Copenhagen strain of vaccinia virus-the common smallpox vaccine-has a higher physical containment level; namely, BSL2. Accordingly, the art has recognized that NYVAC, ALVAC and TROVAC have a lower pathogenicity than any other poxvirus.

Another attenuated poxvirus of preferred use for this invention is Modified Vaccinia virus Ankara (MVA), which acquired defects in its replication ability in humans, as well as most mammalian cells, following over 500 serial passages in chicken fibroblasts (see, e.g., Mayr et al., Infection 3:6-14 (1975); Carrol, M. and Moss, B. Virology 238:198-211 (1997)). MVA retains its original immunogenicity and its variola-protective effect and no longer has any virulence and contagiousness for animals and humans. As in the case of NYVAC or ALVAC, expression of recombinant protein occurs during an abortive infection of human cells, thus providing a safe, yet effective, delivery system for foreign antigens.

The HIV antigen encoding DNA for insertion into these vectors is any that is known to be an effective antigen for protection against a retrovirus. For HIV these would include nucleic acid that can encode at least one of: HIV1gag(+pro)(IIIB), gp120(MN)(+transmembrane), nef(BRU)CTL, pol(IIIB)CTL, ELDKWA or LDKW epitopes, preferably HIV1gag(+pro)(IIIB), gp120(MN) (+transmembrane), two (2) nef(BRU)CTL and three (3) pol(IIIB)CTL epitopes; or two ELDKWA in gp120 V3 or another region or in gp160. The two (2) nef(BRU)CTL and three (3) pol(IIIB)CTL epitopes are preferably CTL1, CTL2, pol1, pol2 and pol3. In the above listing, the viral strains from which the antigens are derived are noted parenthetically.

DNA Vaccines

As an alternative to a viral vaccine, the nucleic acid can also be introduced into the cells of a patient in an expression vector that is not contained within a viral particle. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and WO 98/04720. Examples of DNA-based delivery technologies include, “naked DNA”, facilitated (bupivicaine, polymers, peptide-mediated) delivery, and cationic lipid complexes or liposomes. The nucleic acids can be administered using ballistic delivery as described, for instance, in Fynan et al., Proc Natl Acad Sci USA. 90:11478-82 (1993) and U.S. Pat. No. 5,204,253 or pressure (see, e.g., U.S. Pat. No. 5,922,687). Using this technique, particles comprised solely of DNA are administered, or in an alternative embodiment, the DNA can be adhered to particles, such as gold particles, for administration.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid.

Any of the conventional vectors used for expression in eukaryotic cells may be used for directly introducing DNA into tissue. Expression vectors containing regulatory elements from eukaryotic viruses are typically used in eukaryotic expression vectors, e.g., SV40 vectors. Other exemplary eukaryotic vectors include pMSG, pAV009/A+, pMTO10/A+, pMAMneo-5, baculovirus pDSVE, and any other vector allowing expression of proteins under the direction of such promoters as the SV40 early promoter, SV40 later promoter, metallothionein promoter, human cytomegalovirus promoter, murine mammary tumor virus promoter, Rous sarcoma virus promoter, polyhedrin promoter, or other promoters shown effective for expression in eukaryotic cells.

For example, DNA vaccines can be made from the pVR1332 plasmid (Vical, San Diego, Calif.). This plasmid can be used as a backbone to construct Rev-independent SIV-gag and SIV-env expression vectors. These vectors can then be utilized as DNA vaccines. Briefly, the SIV-gag expression vector can constructed so that a CMV promoter (without introns), the RNA-optimized SIV p57 gag coding region, and the bovine growth hormone polyadenylation site are operably linked. To optimize for RNA expression, the gag inhibitory sequences (INS) can be mutated by introducing multiple silent point mutations that do not affect the encoded protein precursor, as previously described for HIV-1 gag (Qui et al., J. Virol., 73:9145 (1999); Schneider et al., J. Virol., 71:4892 (1997); Schwartz et al., J. Virol., 66:7176)). The SIV-env expression vector can include an RNA optimized gp160 gene which contains 29 point mutations to eliminate the Rev-responsive elements (von Gegerfelt et al., Virology, 232:291 (1997)), and that is conjugated at the 3′ untranslated region to the constitutive transport element of simian retrovirus type 1, which further promotes mRNA export (Bray et al., Proc. Natl. Acad. Sci., 91:1256 (1994), Zolotukhin et al., J. Virol., 68:7944 (1994)).

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using gel electrophoresis or other methods. DNA plasmid preparations of a clinical-grade quality can also be produced through use of commercially available products (Qiagen, Hilden, Germany).

Purified plasmid DNA can be prepared for injection using a variety of formulations. The simplest of these is reconstitution of lyophilized DNA in sterile phosphate-buffer saline (PBS). This formulation, known as “naked DNA,” is particularly suitable for intramuscular (IM) or intradermal (ID) administration.

To maximize the immunotherapeutic effects of plasmid DNA vaccines, alternative methods for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7):682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

Selection of an HIV Specific Epitope

Highly antigenic epitopes for provoking an immune response selective for a specific retroviral pathogen are known. The retrovirus, HIV is a major problem in the United States and in the world. With minor exceptions, the following discussion of HIV epitopes is applicable to other retroviruses except for the differences in sizes of the respective viral proteins. HIV-specific epitopes fall into two major categories, structural and non-structural proteins. Epitopes can be selected from either or both groups of proteins. Structural proteins are a physical part of the virion. Non-structural proteins are regulatory proteins. The envelope is a preferred source of epitopes and gp160, as well as the gp120 and gp41 products thereof, are sources of immunoprotective proteins. Both B and T cell epitopes have been described in the literature and can be used. Peptides selected from the V3 loop of the HIV envelope proteins are of preferred use. In addition other structural proteins have been reported to be immunoprotective including p41, p17 and the gag protein. Non-structural genes include the rev, tat, nef, vif, and vpr genes.

### Patients

The vaccine regimen is typically delivered to patients who are at an early stage of retroviral infection, e.g., HIV-1, or who have undergone anti-retroviral therapy. A preferred patient population of retrovirally infected persons are those that exhibit repeated and sustained proliferative T-cell responses to envelope epitopes, e.g., HIV gp120. More preferred are those patients that also respond to the gag epitopes, e.g. HIV p24. Typically these patients are identified by measuring the ability of their lymphocytes to proliferate in responses to highly purified antigen. In brief, peripheral blood monocytes (PBMC) are collected and cultured in the absence of IL-2 and in the presence of 10 μg of highly purified antigen. After four days the cultures are harvested and proliferation is measured by uptake of radioactive thymidine.

An alternative means of identifying these patients is to use a skin test. Skin tests involve the detection of a delayed type hypersensitive response (DTH) by means of injecting or scratching antigen beneath the surface of the skin. The reaction is measured by the ability or inability of a patient to exhibit hypersensitive response to an aqueous solution of a gp120 or p24 antigen.

Approximately, 1-20 μg is applied. The reaction is determined by measuring wheal sizes from about 24 to about 72 hours after administration of a sample, and more preferably from about 48 hours to about 72 hours after administration of a sample. Preferred wheal sizes for evaluation of the hypersensitivity of an animal range from about 16 mm to about 8 mm, more preferably from about 15 mm to about 9 mm, and even more preferably from about 14 mm to about 10 mm in diameter.

### Highly Active Anti-Retroviral Therapy (HAART)

Antiviral retroviral treatment typically involves the use of two broad categories of therapeutics. They are reverse transcriptase inhibitors and protease inhibitors. There are two type of reverse transcriptase inhibitors: nucleoside analog reverse transcriptase inhibitors and non-nucleoside reverse transcriptase inhibitors. Both types of inhibitors block infection by blocking the activity of the HIV reverse transcriptase, the viral enzyme that translates HIV RNA into DNA which can later be incorporated into the host cell chromosomes.

Nucleoside and nucleotide analogs mimic natural nucleotides, molecules that act as the building blocks of DNA and RNA. Both nucleoside and nucleotide analogs must undergo phosphorylation by cellular enzymes to become active; however, a nucleotide analog is already partially phosphorylated and is one step closer to activation when it enters a cell. Following phosphorylation, the compounds compete with the natural nucleotides for incorporation by HIV's reverse transcriptase enzyme into newly synthesized viral DNA chains, resulting in chain termination.

Examples of anti-retroviral nucleoside analogs include: AZT, ddI, ddC, d4T, Abacavir, Tenofovir, 3TC in combination with AZT (Combivir), and 3TC in combination with AZT and Abacavir (Trizivir).

Nonnucleoside reverse transcriptase inhibitors (NNRTIs) are a structurally and chemically dissimilar group of antiretroviral compounds. They are highly selective inhibitors of HIV-1 reverse transcriptase. At present these compounds do not affect other retroviral reverse transcriptase enzymes such as hepatitis viruses, herpes viruses, HIV-2, and mammalian enzyme systems. They are used effectively in triple-therapy regimes. Examples of NNRTIs are Delavirdine, Efavirenz, and Nevirapine which have been approved for clinical use in combination with nucleoside analogs for treatment of HIV-infected adults who experience clinical or immunologic deterioration. A detailed review can be found in “Nonnucleoside Reverse Transcriptase Inhibitors” AIDS Clinical Care (10/97) Vol. 9, No. 10, p. 75.

Proteases inhibitors are compositions that inhibit HIV protease, which is virally encoded and necessary for the infection process to proceed. Clinicians in the United States have a number of clinically effective proteases to use for treating HIV-infected persons. These include: SAQUINAVIR (Invirase); INDINAVIR (Crixivan); RITONAVIR (Norvir), NELFINAVIR (Viracept®), AMPRENAVIR (Agenerase®), and a combination of LOPINAVIR (Kaletra®) and RITONAVIR (Norvir®).

### CD4⁺ T Cell Counts

To assess a patient's immune system before antiviral treatment and after treatment as well as to determine if the claimed vaccine regimen is working it is important to measure CD4⁺ T-cell counts. A detailed description of this procedure was published by Janet K. A. Nicholson, Ph.D. et al. 1997 Revised Guidelines for Performing CD4⁺ T-Cell Determinations in Persons Infected with Human Immunodeficiency Virus (HIV) in The Morbidity and Mortality Weekly Report, 46(RR-2):[inclusive page numbers], Feb. 14, 1997, Centers for Disease Control.

In brief, most laboratories measure absolute CD4⁺ T-cell levels in whole blood by a multi-platform, three-stage process. The CD4⁺ T-cell number is the product of three laboratory techniques: the white blood cell (WBC) count; the percentage of WBCs that are lymphocytes (differential); and the percentage of lymphocytes that are CD4⁺ T-cells. The last stage in the process of measuring the percentage of CD4⁺ T-lymphocytes in the whole-blood sample is referred to as “immunophenotyping by flow cytometry.

Immunophenotyping refers to the detection of antigenic determinants (which are unique to particular cell types) on the surface of WBCs using antigen-specific monoclonal antibodies that have been labeled with a fluorescent dye or fluorochrome (e.g., phycoerythrin [PE] or fluorescein isothiocyanate [FITC]). The fluorochrome-labeled cells are analyzed by using a flow cytometer, which categorizes individual cells according to size, granularity, fluorochrome, and intensity of fluorescence. Size and granularity, detected by light scattering, characterize the types of WBCs (i.e., granulocytes, monocytes, and lymphocytes). Fluorochrome-labeled antibodies distinguish populations and subpopulations of WBCs.

Systems for measuring CD4⁺ cells are commercially available. For example Becton Dickenson's FACSCount System automatically measure absolutes CD4⁺, CD8⁺, and CD3⁺ T lymphocytes. It is a self-contained system, incorporating instrument, reagents, and controls.

### Viral Titer (Load)

There are a variety of ways to measure viral titer in a patient. A review of the state of this art can be found in the Report of the NIH To Define Principles of Therapy of HIV Infection as published in the; Morbidity and Mortality Weekly Reports, Apr. 24, 1998, Vol 47, No. RR-5, Revised Jun. 17, 1998. It is known that HIV replication rates in infected persons can be accurately gauged by measurement of plasma HIV concentrations.

HIV RNA in plasma is contained within circulating virus particles or virions, with each virion containing two copies of HIV genomic RNA. Plasma HIV RNA concentrations can be quantified by either target amplification methods (e.g., quantitative RT polymerase chain reaction [RT-PCR], Amplicor HIV Monitor assay, Roche Molecular Systems; or nucleic acid sequence-based amplification, [NASBA®], NucliSens™ HIV-1 QT assay, Organon Teknika) or signal amplification methods (e.g., branched DNA [bDNA], Quantiplex™ HIV RNA bDNA assay, Chiron Diagnostics). The bDNA signal amplification method amplifies the signal obtained from a captured HIV RNA target by using sequential oligonucleotide hybridization steps, whereas the RT-PCR and NASBA® assays use enzymatic methods to amplify the target HIV RNA into measurable amounts of nucleic acid product. Target HIV RNA sequences are quantitated by comparison with internal or external reference standards, depending upon the assay used.

Measurements of CD8⁺ Responses

CD8⁺ T-cell responses can be measured, for example, by using tetramer staining of fresh or cultured PBMC (see, e.g., Altman, J. D. et al., Proc. Natl. Acad. Sci. USA 90:10330, 1993; Altman, J. D. et al., Science 274:94, 1996), or γ-interferon release assays such as ELISPOT assays (see, e.g., Lalvani, A. et al., J. Exp. Med. 186:859, 1997; Dunbar, P. R. et al., Curr. Biol. 8:413, 1998; Murali-Krishna, K. et al., Immunity 8:177, 1998), or by using functional cytotoxicity assays. Each of these assays are well-known to those of skill in the art.

For example, a cytotoxicity assay can be performed as follows. Briefly, peripheral blood lymphocytes from patients are cultured with HIV peptide epitope at a density of about five million cells per milliliter. Following three days of culture, the medium is supplemented with human IL-2 at 20 units per milliliter and the cultures are maintained for four additional days. PBLs are centrifuged over Ficoll-Hypaque and assessed as effector cells in a standard ⁵¹Cr-release assay using U-bottomed microtiter plates containing about 10⁴ target cells with varying effector cell concentrations. All cells are assayed twice. Autologous B lymphoblastoid cell lines are used as target cells and are loaded with peptide by incubation overnight during ⁵¹Cr labeling. Specific release is calculated in the following manner: (experimental release−spontaneous release)/(maximum release−spontaneous release)×100. Spontaneous release is generally less than 20% of maximal release with detergent (2% Triton X-100) in all assays.

### Lymphocyte Proliferation Assay

Antigen-specific proliferation can be measured using fresh peripheral blood mononuclear cells (PBMC) isolated by density gradient centrifugation on Ficoll lymphocyte separation medium (ICN Pharmaceuticals, Aurora, Ohio). The cells can be resuspended in RPMI 1640 medium (Life Technologies, Gaithersburg, Md.) containing 5% inactivated human A/B serum (Sigma-Aldrich, St. Louis, Mo.), and cultured at 105 cells per well in triplicates for 3 days in the absence or presence of native HPLC-purified SIV p27 gag or gp120 Env proteins (Advanced BioScience Laboratories, Rockville, Md.) or Con A as a positive control. The cells can then be pulsed overnight with 1 μCi of [³H]thymidine before harvest. The relative rate of lymphoproliferation can be calculated as fold of thymidine incorporation into cellular DNA over medium control (stimulation index (SI)).

### ELISPOT Assay

Monkey IFN—specific ELISPOT kits manufactured by U-Cytech (Utrecht, The Netherlands) can be used to detect the number of cells producing IFN—upon in vitro stimulation. Ninety-six-well flat-bottom plates can be coated with anti-IFN-mAb MD-1 overnight at 4° C. and blocked with 2% BSA in PBS for 1 h at 37° C. Cells (10⁵ per well) can be loaded in quadruplicates in RPMI 1640 containing 5% human serum and 10 μg/ml of a specific peptide or 5 μg/ml Con A as a positive control. The plates can then be incubated overnight at 37° C., 5% CO₂, and developed according to the manufacturer's guidelines (U-Cytech).

### Formulation of Vaccines and Administration

The administration procedure for recombinant virus or DNA is not critical. Vaccine compositions (e.g., compositions containing the poxvirus recombinants or DNA) can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular patient, and the route of administration.

In therapeutic applications, the vaccines are administered to a patient in an amount sufficient to elicit a therapeutic effect, i.e., a CD8⁺, CD4⁺, and/or antibody response to the HIV-1 antigens or epitopes encoded by the vaccines that cures or at least partially arrests or slows symptoms and/or complications of HIV infection. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered in any combination. In preferred embodiments a DNA HIV vaccine is administered to a patient one or more times and is followed by delivery of one or more administrations of a recombinant pox virus HIV vaccine. The recombinant viruses are typically administered in an amount of about 10⁴ to about 10⁹ pfu per inoculation; often about 10⁴ pfu to about 10⁶ pfu. Higher dosages such as about 10⁴ pfu to about 10¹⁰ pfu, e.g., about 10⁵ pfu to about 10⁹ pfu, or about 10⁶ pfu to about 10⁸ pfu, can also be employed. For example, a NYVAC-HIV vaccine can be inoculated by the intramuscular route at a dose of about 10⁸ pfu per inoculation, for a patient of 170 pounds.

Suitable quantities of DNA vaccine, e.g., plasmid or naked DNA can be about 1 μg to about 100 mg, preferably 0.1 to 10 mg, but lower levels such as 0.1 to 2 mg or 1-10 μg can be employed. For example, an HIV DNA vaccine, e.g., naked DNA or polynucleotide in an aqueous carrier, can be injected into tissue, e.g., intramuscularly or intradermally, in amounts of from 10 μl per site to about 1 ml per site. The concentration of polynucleotide in the formulation is from about 0.1 μg/ml to about 20 mg/ml.

The vaccine may be delivered in a physiologically compatible solution such as sterile PBS in a volume of, e.g., one ml. The vaccines may also be lyophilized prior to delivery. As well known to those in the art, the dose may be proportional to weight.

The compositions included in the vaccine regimen can be administered alone, or can be co-administered or sequentially administered with other immunological, antigenic, vaccine, or therapeutic compositions. These include adjuvants, and chemical or biological agent given in combination with, or recombinantly fused to, an antigen to enhance immunogenicity of the antigen. Such other compositions can also include purified antigens from the immunodeficiency virus or from the expression of such antigens by a second recombinant vector system which is able to produce additional therapeutic compositions. For examples, these compositions can include a recombinant poxvirus which expresses other immunodeficiency antigens or biological response modifiers (e.g., cytokines or co-stimulating molecules, further discussed below). Examples of adjuvants which also may be employed include Freund's complete adjuvant and MPL-TDM adjuvant (monophosphoryl Lipid A, synthetic trehalose dicorynomycolate). Again, co-administration is performed by taking into consideration such known factors as the age, sex, weight, and condition of the particular patient, and, the route of administration.

Compositions that may also be administered with the vaccines include other agents to potentiate or broaden the immune response. Examples of these agents include cytokines, such as interleukin-2 (IL-2), interleukin-7 (IL-7), interleukin-12 (IL-12), interleukin-15 (IL-15), macrophage colony stimulating factor (GM-CSF), and interferon beta (IFNβ). Agents may also be selected from other types of molecules known to potentiate or broaden the immune response, such as the CD40 ligand. These agents can be administered at specified intervals of time, or continuously administered (see, e.g., Smith et al., N Engl J Med 1997 Apr. 24; 336(17):1260-1; and Smith, Cancer J Sci Am. 1997 Dec.; 3 Suppl 1:S 137-40). For example, IL-2 can be administered in a broad range, e.g., from 10,000 to 1,000,000 or more units. Administration can occur continuously following vaccination. Often, low doses, e.g. 100,000 to 200,000, often 120,000, 150,000 or 170,000, units of IL-2 can be particularly useful. In addition, these agents may be encoded on plasmids that are coadministered with a DNA vaccine. Methods for such administration have been reported (Kwissa et al., J. Mol. Med., 81:91 (2003); Nobiron et al., Vaccine, 21:2091 (2003); Boyer et al., J. Liposome Res., 12:137 (2002).

The vaccines can additionally be complexed with other components such as peptides, polypeptides and carbohydrates for delivery. For example, expression vectors, i.e., nucleic acid vectors that are not contained within a viral particle, can be complexed to particles or beads that can be administered to an individual, for example, using a vaccine gun.

Nucleic acid vaccines are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

For example, naked DNA or polynucleotide in an aqueous carrier can be injected into tissue, such as muscle, in amounts of from 10 μl per site to about 1 ml per site. The concentration of polynucleotide in the formulation is from about 0.1 μg/ml to about 20 mg/ml.

Vaccines can be delivered via a variety of routes. Typical delivery routes include parenteral administration, e.g., intradermal, intramuscular or subcutaneous routes. Other routes include oral administration, intranasal, and intravaginal routes.

The expression vectors of use for the invention can be delivered to the interstitial spaces of tissues of a patient (see, e.g., Felgner et al., U.S. Pat. Nos. 5,580,859, and 5,703,055). Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5,187,074.

Examples of vaccine compositions of use for the invention include liquid preparations, for orifice, e.g., oral, nasal, anal, vaginal, etc. administration, such as suspensions, syrups or elixirs; and, preparations for parenteral, subcutaneous, intradermal, intramuscular or intravenous administration (e.g., injectable administration) such as sterile suspensions or emulsions. In such compositions the recombinant poxvirus, expression product, immunogen, DNA, or modified gp120 or gp160 can be in admixture with a suitable carrier, diluent, or excipient such as sterile water, physiological saline, glucose or the like.

The vaccines can be incorporated, if desired, into liposomes, microspheres or other polymer matrices (see, e.g., Felgner et al., U.S. Pat. No. 5,703,055; Gregoriadis, Liposome Technology, Vols. I to III (2nd ed. 1993). Liposomes, for example, which consist of phospholipids or other lipids, are nontoxic, physiologically acceptable and metabolizable carriers that are relatively simple to make and administer. Liposomes include emulsions, foams, micelles, insoluble monolayers, liquid crystals, phospholipid dispersions, lamellar layers and the like.

Liposome carriers can serve to target a particular tissue or infected cells, as well as increase the half-life of the vaccine. In these preparations the vaccine to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired immunogen of the invention can be directed to the site of lymphoid cells, where the liposomes then deliver the immunogen(s).

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

### Administration of Neutralizing Antibodies, Agents that Block Virus Receptor Binding, or Cytotoxic Immunoconjugates to Control Viremia

Neutralizing Antibodies and Agents that Block Receptor Binding

In the methods of the invention, the vaccine regimens comprise a step of controlling viremia through administering agents, typically neutralizing antibodies, that block binding of the virus to its receptor. Antibodies that neutralize HIV are well known to those of skill in the art. The primary receptor for the human immunodeficiency virus type 1 (HIV-1) is the CD4 molecule, found predominantly on the surface of T-lymphocytes. The binding of HIV-1 to CD4 occurs via the major viral envelope glycoprotein gp120 and initiates the viral infection process. Numerous groups have reported the preparation of antibodies, e.g., human monoclonal antibodies, that neutralize HIV isolates in vitro. The antibodies typically have immunospecificities for epitopes on the HIV glycoprotein gp160 or the related glycoproteins gp120 or gp41. See, for example Karwowska et al., Aids Research and Human Retroviruses, 8:1099-1106 (1992); Takeda et al., J. Clin. Invest., 89:1952-1957 (1992); Tilley et al., Aids Research and Human Retroviruses, 8:461-467 (1992); Laman et al., J. Virol., 66:1823-1831 (1992); Thali et al., J. Virol., 65:6188-6193 (1991); Ho et al., Proc. Natl. Acad. Sci., USA, 88:8949-8952 (1991); D'Souza et al., AIDS, 5:1061-1070 (1991); Tilley et al., Res. Virol., 142:247-259 (1991); Broliden et al., Immunol., 73:371-376 (1991); Matour et al., J. Immunol. 146:4325-4332 (1991); and Gomy et al., Proc. Natl. Acad. Sci., USA, 88:3238-3242 (1991). Other neutralizing antibodies are described for example in U.S. Pat. Nos. 5,804,440 and 5,961,976.

An example of a neutralizing antibody is an anti-gp120 recombinant human monoclonal antibody (MAb) designated IgG1 b12 that has specificity for the gp120 binding site for CD4 (anti gp120 CD4-BS) (Burton et al., Science, 266:1024-1027, 1994). IgG1 b12 was generated as an Fab fragment from an antibody-phage display library prepared from bone marrow of a long-term asymptomatic HIV-1 seropositive donor and was converted to a whole human antibody by cloning into a recombinant DNA IgG1 expression vector.

Other HIV-1 neutralizing antibodies that can be used include the human monoclonal antibodies 2F5 and 2G12 (see, e.g., AIDS 2002 Jan. 25; 16(2):227-33), the CD4-immunoglobulin G2 (IgG2) (see, e.g., Gauduin et al., J Virol 1998 Apr.; 72(4):3475-8)

Neutralizing antibodies that target the HIV co-receptors, CXCR4 and CCR5, may also be useful as blocking agents. Such antibodies typically bind to the co-receptor or the region of the HIV envelope that binds to the co-receptor. PRO 140 is an example of a monoclonal antibody that binds to CCR5 (Olson et al., Program and Abstracts of the 40th Interscience Conference on Antimicrobial Agents and Chemotherapy, Toronto, Canada, September 200, Abstract 500).

Additional neutralizing antibodies can also be generated. Techniques to generate antibodies are well known to those of skill in the art. Virus neutralization can then be measured by a variety of in vitro and in vivo methodologies. To determine whether a particular antibody inhibits infection of CD4-expressing cells by HIV, any indication of HIV infection could be monitored. Useful in vitro indicators of HIV infection include, for example, secretion of HIV core antigen p24. Preferably, inhibition of HIV infection is determined by comparing HIV p24 levels in the presence and absence of the antibody in HIV-infected CD4-expressing cell cultures. Another exemplary method is an in vitro assay that measures inhibition of HIV-induced syncytia formation. For example, to evaluate the ability of a particular antibody to block HIV-induced syncytia formation among CD4-expressing cells, any known syncytia assay may be used. Preferably, a primary isolate or HIV-infected CD4-expressing tissue culture cells (e.g., H9) are added to cultures of MT-2 cells. Varying amounts of the antibodies are then added. Negative controls are supplemented with regular culture medium, or with an irrelevant antibody in the presence of HIV. A positive control with giant syncytia inducing strains may also be used. After incubation, all of the cultures are scored by visual quantification of syncytia or plaque formation, in the case of giant syncytia inducing strains. In this way, the ability of an antibody to block syncytia formation or to reduce the number of plaques formed in a culture is scored.

Additional Blocking Agents

Additional agents that inhibit HIV receptor binding may also be used in this invention. Such agents include, for example, peptides that bind to the virus receptor, or one or more viral coat proteins and prevent the protein from binding to the receptor. Many of these agents are known as HIV entry inhibitors. The receptor can be either CD4 or a viral co-receptor, e.g., CCR5, or CXCR4. For example, T-20 is a synthetic peptide that corresponds to 36 amino acids within the C-terminal heptad repeat region (HR2) of human immunodeficiency virus type 1 (HIV-1) gp41. This peptide is thought to exert their antiviral activity by interfering with the conformational changes that occur within gp41 to promote membrane fusion following gp120 interactions with CD4 and coreceptor molecules (e.g., J Virol 2001 September; 75(18):8605-14). Such a peptide may be administered as a blocking agent. Another agent is PRO 542 (Progenics Pharmaceuticals, Tarrytown, N.Y.). PRO 542 is a recombinant antibody-like fusion protein wherein the D1D2 domains of human CD4 are grafted onto the heavy and light chain constant regions of human IgG2. PRO 542 binds to gp120 displayed on the surface of an HIV virion and thereby neutralizes the virion. Fragments of gp41, such as DP-178, may be used to block HIV entry into a cell (Kilby et al., Nature Med., 4:1302 (1998)). RANTES is another agent that may be used to block entry of HIV into a cell. RANTES is a 68 amino acid peptide that binds to the CCR5 receptor. A RANTES analog may also be used to block entry of HIV into a cell (Mosier et al., J. Virol., 73:3544 (1999)). TAK-779 is an example of an inhibitor of attachment of HIV-1 to CCR5 (Tremblay et al., Program and Abstracts of the 40th Interscience Conference on Antimicrobial Agents and Chemotherapy, Toronto, Canada, September 200, Abstract 1164). AMD-3100 and AMD-070 are examples of compounds that act on the CXCR4 receptor (Schols et al., Program and Abstracts of the 7th Conference of Retroviruses and Opportunistic Infections, San Francisco, Calif., January-February, 2000, Abstract S18; AnorMED, Langley, British Columbia, Canada).

Derivatives of the aforementioned blocking agents may also be used within the invention. For example, analogs of peptides such as RANTES, T-20, and DP-178 may be prepared that contain one or more non-peptide bonds. Such analogs are commonly known as peptidomimetics. An advantage of peptidomimetics is that they offer increased resistance to proteases that act to decrease the lifetime of therapeutic peptides. Methods to prepare peptidomimetics are known in the art (Fauchere, Adv. Drug Res., 15: 29 (1986); Evans et al., J. Med. Chem., 30:1229 (1987)). Blocking agents that may be used within the invention may also be modified to include greater of fewer amino acids than are present in the aforementioned blocking agents, as long as they act to block entry of HIV into a cell.

Other agents include small molecules that bind to the receptor or co-receptor. For example, SCH-C (SCH 351125), an orally available, small molecule inhibitor of HIV-1 entry via the CCR5 coreceptor (see, e.g., Stirzki et al., Proc Natl Acad Sci USA 2001 Oct. 23; 98.). Such an agent may also be administered either in lieu of a neutralizing antibody or in addition to a neutralizing antibody.

Immunoconjugates

Immunoconjugates that target HIV-infected cells may also be used as a component of the therapeutic regimen, either in place of, or in addition to, a blocking agent or neutralizing antibody. Immunoconjugates for use in the invention comprise an antibody to an HIV envelope protein and a cytotoxic component. The components may be directly adjoined or may be joined through a linker. The antibody may be, but need not be, a neutralizing antibody. The cytotoxic component of the immunoconjugate may be any agent that kills an HIV-infected cells. Such agents include, but are not limited to, a toxin, e.g., a Pseudomonas exotoxin, diphtheria toxin, and the lice; an RNAse, such as an RNAse A family member; a tag such as a radio-label, which is toxic to the cell; or a small molecule.

The toxic moiety and the antibody may be conjugated by chemical or by recombinant means. Chemical modifications include, for example, derivitization for the purpose of linking the moieties to each other, either directly or through a linking compound, by methods that are well known in the art of protein chemistry. For example, the toxic moiety may be linked to the recognition moiety via a heterobifunctional coupling reagent that ultimately contributes to formation of an intermolecular disulfide bond between the two moieties. Other types of coupling reagents that are useful in this capacity for the present invention are described, for example, in U.S. Pat. No. 4,545,985. Alternatively, an intermolecular disulfide may conveniently be formed between cysteines in each moiety which occur naturally or are inserted by genetic engineering. The means of linking moieties may also use thioether linkages between heterobifunctional crosslinking reagents or specific low pH cleavable crosslinkers or specific protease cleavable linkers or other cleavable or noncleavable chemical linkages. The means of linking moieties of the immunotoxins may also comprise a peptidyl bond formed between moieties which are separately synthesized by standard peptide synthesis chemistry or recombinant means.

Possible chemical modifications of the protein moieties of the present invention also include derivitization with polyethylene glycol (PEG) to extend time of residence in the circulatory system and reduce immunogenicity, according to well known methods (See, for example, Lisi, et al., Applied Biochem. 4:19 (1982); Beauchamp, et al., Anal. Biochem. 131:25 (1982); and Goodson, et al., Bio/Technology 8:343 (1990)).

Immunoconjugates can also be produced using recombinant DNA techniques, for example, to form a single chain that comprises the relevant functional domains.

Methods of producing recombinant fusion proteins are well known to those of skill in the art. Thus, for example, Chaudhary, et al., Nature 339:394 (1989); Batra, et al., J. Biol. Chem. 265:15198 (1990); Batra, et al., Proc. Nat'l Acad. Sci. USA 86:8545 (1989); Chaudhary, et al., Proc. Nat'l Acad. Sci. USA 87:1066 (1990), describe the preparation of various single chain antibody-toxin fusion proteins. Expression constructs may be prepared using well known techniques (see, e.g., Russell & Sambrook, Molecular Cloning, Laboratory Manual (3rd ed. 2001); and Current Protocols in Molecular Biology (Ausubel et al., eds., 1994-2001)).

Administration and Formulation of Blocking Agents and Immunoconjugates

Agents that block HIV infectivity, e.g., neutralizing antibodies, or other agents that block virus binding to its receptor (such as peptides, small organic molecules and the like) or cytotoxic immunoconjugates, are administered following vaccination and typically several days, e.g., 1, 2, 3, 4, 5, or more days, prior to the cessation of anti-retroviral therapy. The agents may be administered more than once and in any combination. The agents that block HIV infectivity are administered in an amount that reduces viral load by at least 50%, preferably 75%.

The agents that block HIV infectivity, e.g., neutralizing antibodies, blocking peptides or small molecules, and cytotoxic immunoconjugates, are administered using techniques well known in the art. Pharmaceutical compositions typically comprise a pharmaceutically acceptable carrier as described above. Single or multiple administrations of the compositions may be administered depending on the dosage and frequency as required and tolerated by the patient. In any event, the composition should provide a sufficient quantity of the therapeutic agent to effectively treat the patient.

Preferably, the agents that block HIV infectivity for administration will commonly comprise a solution of the neutralizing antibody or other blocking agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of protein in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.01 to 100 mg per patient per day. Dosages from 0.1 up to about 1000 mg per patient per day may be also be used. Methods for preparing compositions will be known or apparent to those skilled in the art and are described in more detail in such publications as REMINGTON'S PHARMACEUTICAL SCIENCE, 15TH ED., Mack Publishing Co., Easton, Pa., (1980).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill will readily recognize a variety of noncritical parameters which could be changed or modified to yield essentially similar results.

### Example 1

### Protocol for the Treatment of an HIV-Infected Individual Who is Treated with Anti-Retroviral Therapy

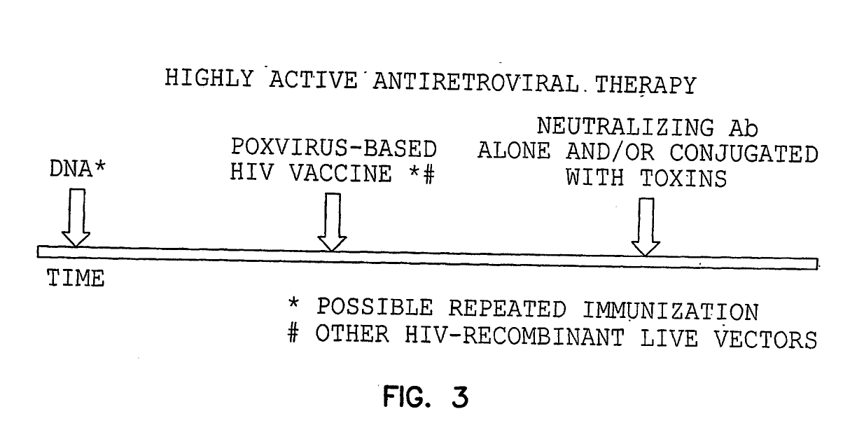

An illustration of administration of the treatment protocol of the invention is provided in FIG. 3. For example, an individual with 400 or more CD4⁺ T-cells/ml would be treated with HAART for a minimum of 2-3 months. DNA-HIV vaccination (up to 10 mg) would then be given, usually intramuscularly one or more times in presence of an optimal adjuvant. At the time of plateau of the immune response measured in blood (i.e., measurements of CD4⁺ and CD8⁺ responses), one or more inoculations of a poxvirus HIV vaccine would be administered intramuscularly at a dose ranging between 10⁸ to 10⁹. Again, when the immune response plateaus, the administration of substances able to block the infectivity of HIV will be started. Such a substance may be, for example, a cytotoxic immunoconjugate specific for HIV-infected cells, a neutralizing antibody, or other agent that blocks virus-receptor binding.

After an interval of days, typically 2-3 days, (the optimal plasma concentration will be measured in preclinical studies for each of the agents used), HAART will be suspended. Plasma virus levels will be measured using state of the art techniques. The substance will be maintained for a minimum of 2-3 weeks. at the end of this time the treatment will be suspended and plasma virus levels monitored. In case virus level exceed 10⁴ to 10⁵ copies per ml, the treatment can be repeated from the beginning, or variations thereof can be used.

### Example 2

### Animals and Administration Protocols

### Animals

All animals will be colony-bred rhesus macaque monkeys (Macaca mulatta) obtained from Covance Research Products (Alice, Tex.). The monkeys will be housed and handled in accordance with the standards of the Association for the Assessment and Accreditation of Laboratory Animal Care International. All monkeys will initially be seronegative for SIV-1, simian T cell leukemia virus type 1, and herpesvirus B. All monkeys will be screened for the presence of the Mamu-A*01 allele by PCR and the amplified DNA will be sequenced to confirm the Mamu-A*01 status.

### Inoculation with Pathogenic SIVmac251

A monkey will be inoculated intravenously with 10 TCID₅₀ of pathogenic SIVmac251.

### Anti-Retroviral Treatment (ART)

A monkey will receive subcutaneous injections of 20 mg/kg/day of PMPA ((R)-9-(2-phosphonylmethoypropyl)adenine), oral administrations of 1.2 mg/kg/day of Stravudine (d4T) divided into 2 doses daily, and intravenous administration of 10 mg/kg/day of dideoxyinosine (ddI).

### Immunizations

A monkey will be immunized intramuscularly with 10⁸ plaque forming units (PFU) of mock ALVAC or ALVAC-SIV vaccine.

For DNA immunization, 4 mg of each plasmid (SIV-env and SIV-gag) will be administered. Four doses of 0.75 mg of each plasmid will be injected intramuscularly into a monkey at two sites on each leg; five doses of 0.2 mg of each plasmid will be injected intradermally at five different sites in the abdominal area of a monkey.

### Administration of Neutralizing Antibody CD4-Ig2

The neutralizing antibody, CD4-Ig2, will be administered to a monkey through intravenous injection at a dosage of 10 mg/kg every two days before and during HAART suspension. CD4-Ig2 will be administered eleven times.

### Administration of IL-7 and IL-15

IL-7 will be administered to a monkey subcutaneously every three days following the start of administration at a dose of 100 μg/Kg until the end of that administration session. In total, IL-7 will be administered eight times per administration session.

IL-15 will be administered to a monkey subcutaneously every fourth day following the start of administration at a dose of 10 μg/kg until the end of that administration session. In total, IL-15 will be administered seven times per administration session

### Example 3

### Administration of DNA Vaccines, a Live Virus Vaccine, and Neutralizing Antibody to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four and thirty, each monkey will be immunized with the DNA vaccines (SIV-env and SIV-gag) according to the above described procedure. At week thirty-six, each monkey will be immunized with viral vaccine (ALVAC-SIV) according to the procedure described herein. At week forty, the neutralizing antibody (CD4-Ig2) will be administered to each monkey according to the protocol described herein. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 4

### Administration of a Live Virus Control Vaccine (Mock Vaccine) to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with a control viral vaccine (ALVAC) that lacks the SIV gag, pol, and env genes. Immunization will be according to the procedure described herein for ALVAC-SIV. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 5

### Administration of a Live Virus Control Vaccine (Mock Vaccine) and a Neutralizing Antibody to Simian Immunodeficiency Virus (SIV) Infected Monkeys Undergoing Anti-Retroviral Treatment

Monkeys will be inoculated intravenously with 10 TCID₅₀ of pathogenic SiVmac251 on week zero. On week sixteen, each monkey will begin anti-retroviral treatment (ART) according to the above described procedure. On weeks twenty-four, thirty, and thirty-six, each monkey will be immunized with a control viral vaccine (ALVAC) that lacks the SIV gag, pol, and env genes. Immunization will be according to the procedure described herein for ALVAC-SIV. At week forty, the neutralizing antibody (CD4-Ig2) will be administered to each monkey according to the protocol described herein. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 6

### Administration of a Live Virus Control Vaccine (Mock Vaccine) and IL-7 to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with a control viral vaccine (ALVAC) that lacks the SIV gag, pol, and env genes. In addition, on weeks twenty-four, thirty, and thirty-six, IL-7 will be administered to each monkey according to the above described protocol. Immunization with ALVAC will be according to the procedure described herein for ALVAC-SIV. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 7

### Administration of a Live Virus Vaccine and IL-7 to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with the viral vaccine (ALVAC-SIV) according to the above described procedure. In addition, on weeks twenty-four, thirty, and thirty-six, IL-7 will be administered to each monkey according to the above described protocol. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 8

### Administration of a Live Virus Control Vaccine (Mock Vaccine) and IL-15 to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with a control viral vaccine (ALVAC) that lacks the SIV gag, pol, and env genes. In addition, on weeks twenty-four, thirty, and thirty-six, IL-15 will be administered to each monkey according to the above described protocol. Immunization with ALVAC will be according to the procedure described herein for ALVAC-SIV. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 9

### Administration of a Live Virus Vaccine and IL-15 to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with the viral vaccine (ALVAC-SIV) according to the above described procedure. In addition, on weeks twenty-four, thirty, and thirty-six, IL-15 will be administered to each monkey according to the above described protocol. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 10

### Administration of DNA Vaccines and a Live Virus Vaccine to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four and thirty, each monkey will be immunized with DNA vaccines (SIV-env and SIV-gag) according to the above described procedure. On week thirty-six, each monkey will be immunized with a viral vaccine (ALVAC-SIV) according to the above described procedure. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

### Example 11

### Administration of a Live Virus Vaccine to Simian Immunodeficiency Virus (SIV) Infected Monkeys Undergoing Anti-Retroviral Treatment

Monkeys will be inoculated intravenously with 10 TCID₅₀ of pathogenic SIVmac251 on week zero. On week sixteen, each monkey will begin anti-retroviral treatment (ART) according to the above described procedure. On weeks twenty-four, thirty, and thirty-six, each monkey will be immunized with the viral vaccine (ALVAC-SIV) according to the above described procedure. At week forty-one, the anti-retroviral treatment will be discontinued for each monkey. The protocol is represented in Table I below.

Blood and tissue samples will be collected from each monkey during the course of administration and tested for viral load, CD8⁺ cell activity, lymphocyte proliferation, and intracellular cytokine staining of CD4⁺ and CD8⁺ T-cells following stimulation with SIV-specific peptides.

TABLE I
Summary of Examples 3-11

| Example | Week 0 | Week 16 | Week 24 | Week 30 | Week 36 | Week 40 | Week 41 |
|---|---|---|---|---|---|---|---|
| 3 | SIVmac251 | ART | DNA | DNA | ALVAC-SIV | CD4-Ig2 | ART off |
| 4 | SIVmac251 | ART | ALVAC | ALVAC | ALVAC | — | ART off |
| 5 | SIVmac251 | ART | ALVAC | ALVAC | ALVAC | CD4-Ig2 | ART off |
| 6 | SIVmac251 | ART | ALVAC + IL-7 | ALVAC + IL-7 | ALVAC + IL-7 | — | ART off |
| 7 | SIVmac251 | ART | ALVAC-SIV + IL-7 | ALVAC-SIV + IL-7 | ALVAC-SIV + IL-7 | — | ART off |
| 8 | SIVmac251 | ART | ALVAC + IL-15 | ALVAC + IL-15 | ALVAC + IL-15 | — | ART off |
| 9 | SIVmac251 | ART | ALVAC-SIV + IL-15 | ALVAC-SIV + IL-15 | ALVAC-SIV + IL-15 | — | ART off |
| 10 | SIVmac251 | ART | DNA | DNA | ALVAC-SIV | — | ART off |
| 11 | SIVmac251 | ART | ALVAC-SIV | ALVAC-SIV | ALVAC-SIV | — | ART off |

What is claimed is:

1. A method to potentiate a CD8⁺ or a CD4⁺ response to a human immunodeficiency virus-I (HIV-1) epitope in a human comprising:
administering at least one nucleic acid vaccine that encodes at least one human immunodeficiency virus-1 epitope to the human; and
administering at least one viral vaccine that encodes at least one human immunodeficiency virus-1 epitope to the human, wherein the nucleic acid vaccine and the viral vaccine can be administered to the human together or in any order.

2. The method of claim 1, further comprising administration of at least one anti-viral agent to the human, wherein the anti-viral agent can be administered together with the viral vaccine and the nucleic acid vaccine, or in any order.

3. The method of claim 1, further comprising administration of at least one agent that reduces human immunodeficiency virus-1 infectivity, wherein the agent can be administered together with the viral vaccine and the nucleic acid vaccine, or in any order.

4. The method according to any one of claims 1-3, further comprising administration of at least one immunoconjugate to the human.

5. The method according to any one of claims 1-3, further comprising administration of at least one cytokine to the human.

6. The method of claim 1, wherein administration of the viral vaccine and the nucleic acid vaccine potentiates a CD8⁺ or CD4⁺ response that is greater in magnitude than administration of the viral vaccine or the nucleic acid vaccine alone.

7. The method of claim 1, wherein the human has a viral load that is less than 10,000 viral copies per milliliter of plasma.

8. The method of claim 1, wherein the human has a viral load that is less than 5,000 viral copies per milliliter of plasma.

9. The method of claim 1, wherein the human has a viral load that is less than 2,000 viral copies per milliliter of plasma.

10. The method of claim 1, wherein the human has a CD4⁺ cell count above 300 cells per milliliter.

11. The method of claim 1, wherein the human has a CD4⁺ cell count above 400 cells per milliliter.

12. The method of claim 1, wherein the human has a CD4⁺ cell count above 500 cells per milliliter.

13. The method of claim 1, wherein the human has a viral load of less than 10,000 viral copies per milliliter of plasma, a CD4⁺ cell count above 400 cells per milliliter, and has been treated with one or more anti-viral agents that lowered viral load and increased CD4⁺ count in the human relative to the viral load and CD4⁺ count in the human before treatment with the anti-viral agent.

14. The method of claim 1, wherein the nucleic acid vaccine encodes at least one structural protein or at least one non-structural protein.

15. The method of claim 14, wherein the structural protein is selected from the group consisting of gp160, gp120, and gp41.

16. The method of claim 14, wherein the structural protein is encoded by a gene selected from the group consisting of env, pol, or gag.

17. The method of claim 14, wherein the non-structural protein is encoded by a gene selected from the group consisting of rev, tat, nef, vif, vpr, and vpu.

18. The method of claim 1, wherein the viral vaccine is a live viral vaccine or dead viral vaccine.

19. The method of claim 1, wherein the viral vaccine is an attenuated recombinant pox virus vaccine.

20. The method of claim 19, wherein the attenuated recombinant pox virus is selected from the group consisting of NYVAC, ALVAC, and fowlpox.

21. The method of claim 3, wherein the agent that reduces human immunodeficiency virus-1 infectivity is selected from the group consisting of an HIV-1 neutralizing antibody, a peptide that binds to an HIV-1 receptor, and a small molecule that binds to an HIV-1 receptor or an HIV-1 coreceptor.

22. The method of claim 21, wherein the HIV-1 neutralizing antibody binds to gp160, gp120, gp41, a CD4 receptor, a CCR5 receptor, or a CXCR4 receptor.

23. The method of claim 21, wherein the HIV-1 neutralizing antibody is selected from the group consisting of IgG b12, 2F5, 2G12, IgG2, and PRO 140.

24. The method of claim 21, wherein the peptide that binds to an HIV-1 receptor is selected from the group consisting of T-20, PRO 542, DP-178, and RANTES.

25. The method of claim 21, wherein the peptide that binds to the small molecule that binds to an HIV-1 receptor or an HIV-1 coreceptor is selected from the group consisting of TAK-779, AMD-3100, AMD-070, and SCH 351125.

26. The method of claim 4, wherein the immunoconjugate comprises a cytotoxic component and an antibody component that binds to HIV-1 protein expressed on an infected cell.

27. The method of claim 26, wherein the cytotoxic component is selected from the group consisting of Pseudomonas exotoxin, diphtheria toxin, an RNAase, and a radio-label.

28. The method of claim 5, wherein the cytokine is selected from the group consisting of interleukin-2, interleukin-7, interleukin-12, interleukin-15, macrophage colony stimulating factor, interferon beta, and CD40 ligand.

29. The method of claim 2, wherein the anti-viral agent is an anti-retroviral agent.

30. The method of claim 29, wherein the anti-retroviral agent is selected from the group consisting of a protease inhibitor, an HIV entry inhibitor, a reverse transcriptase inhibitor, and an anti-retroviral nucleoside analog.

31. The method of claim 1, further comprising administration of at least one anti-viral agent to the human, and at least one agent that reduces human immunodeficiency virus-1 infectivity, wherein the anti-viral agent and the agent that reduces human immunodeficiency virus-1 infectivity can be administered together with the viral vaccine and the nucleic acid vaccine, or in any order.

32. The method according to claim 31, further comprising administration of at least one immunoconjugate to the human.

33. The method according to any one of claims 31 or 32, further comprising administration of at least one cytokine to the human.
